(19) United States
(12) Patent Application Publication
(10) Pub. No.: US 2010/0247687 A1
(43) Pub. Date: Sep. 30, 2010

(54) FORMULATION FOR TATTOO REMOVAL AND METHOD OF USING SAME

(76) Inventor: Mary Arnold-Ronish, Las Vegas, NV (US)

Correspondence Address: Neil S. Goldstein, 4607 Chartley Way NE, Roswell, GA, 30075, US

(21) Appl. No.: 12/413,730

(22) Filed: Mar. 30, 2009

(51) Int. Cl. A61K 36/886

(52) U.S. Cl. 424/744

(57) ABSTRACT

A formulation for tattoo removal and method for using the same are disclosed.

### TECHNICAL FIELD

This invention relates generally to the removal of tattoos and, more specifically, to a formulation for removing tattoos from human skin and a method of using the same.

### BACKGROUND OF THE INVENTION

A tattoo is a body modification made by inserting ink into the layers of skin to change the pigment for decorative or other reasons. The tattooing process involves the placement of pigment into the skin's dermis, the layer of connective tissue underlying the epidermis. After initial injection, pigment is dispersed throughout a homogenized damaged layer down through the epidermis and upper dermis, in both of which the presence of foreign material activates the immune system's phagocytes to engulf the pigment particles. As healing proceeds, the damaged epidermis flakes away (eliminating surface pigment) while granulation tissue forms deeper in the skin, which is later converted to connective tissue by collagen growth. This mends the upper dermis, where pigment remains trapped within fibroblasts, ultimately concentrating in a layer just below the dermis/epidermis boundary. Its presence there is stable, but in the long term (decades) the pigment tends to migrate deeper into the dermis, accounting for the degraded detail of old tattoos.

The most common method of tattooing is through use of an electric tattoo machine, which inserts ink into the skin via a group of needles that are attached to an oscillating unit. The unit rapidly and repeatedly drives the needles in and out of the skin, usually 80 to 150 times a second. A small tattoo of simple design might take fifteen minutes to complete, whereas a more elaborate design may require multiple, lengthy sessions.

A wide range of dyes and pigments can be used in tattoos, from inorganic materials like titanium dioxide and iron oxides to carbon black, azo dyes, and acridine, quinoline, phthalocyanine and naphthol derivates, dyes made from ash, and other mixtures. The current trend for tattoo pigment favors using Acrylonitrile butadiene styrene (ABS plastic) as a colorant. When ground down to an average diameter of slightly less than 1 micrometer, ABS plastics create tattoo pigments that are less likely to fade or blur over time than the traditional pigments.

Recently, cosmetic tattoos have become increasingly popular. It is estimated that as many as 7 in 10 Americans have at least one tattoo. With such increased popularity comes an increased need for tattoo removal. Many of these individuals, however, will, at some point, wish to have their tattoo removed for one of many reasons. For example, an individual may have impulsively elected to receive a tattoo and now regrets that decision. Alternatively, a change in life circumstances may motivate the desire to have a tattoo removed. For example, an individual may elect to receive a tattoo with the name or image of a spouse or lover from whom they are now estranged. Even if an individual desires to keep their tattoo, outside influence may motivate the decision to have it removed. A tattoo in an area of the body not covered by clothing such as the face, neck, hands or lower arms may make securing employment in certain professions more difficult.

Current treatment options for tattoo removal include a variety of lasers, dermabrasion, salabrasion, surgical excision, and cryotherapy. Although some treatments may be effective, they may be expensive, time consuming, and painful. In some cases, such treatments also may result in cosmetically undesirable scarring. One of the more effective tattoo removal treatments is a laser surgical technique in which the tattooed area is irradiated with a high-energy, pulsating laser beam. The tattoo ink pigments absorb a portion of the laser radiation. As a consequence, the pigment particles become sufficiently hot that they decompose into smaller fragments. In the process, the cellular integrity of the surrounding dermal cells may be destroyed. A single laser treatment results in some fading of the tattoo because the human immune system is able to remove some of the pigment fragments; however, most pigment fragments become re-engulfed by still intact dermal cells and so remain visible. In nearly all cases, patients are not satisfied with the results of the first laser treatment and they usually return for additional treatments.

Irradiating the tattooed area with enough energy to fragment the tattoo ink pigments can cause a painful burn to form on the skin. Consequently, laser treatments are sometimes spaced at least one month apart in order to afford the skin time to heal. Often, as many as nine to twenty such treatments may be required to sufficiently fade the tattoo, resulting in substantial pain and financial cost. The laser treatment procedure may be ineffective for removing certain colors and may, instead of removing an image, transform some colors such as, for example, transforming a red lipstick tattoo to a black shade that can be very difficult to remove by laser surgical treatment.

Accordingly, there is a need in the art for a tattoo removal process that is safe (i.e., less damaging to the skin), pain free, efficient, economical and complete and does not subject the patient to excessive pain or discomfort.

### SUMMARY

The present invention provides a method of tattoo removal that includes opening the skin covering a tattoo to access the underlying ink and treating the opened area with a layer of a tattoo removal formulation consisting essentially of sea salt, purified water, aloe barbadensis (aloe vera) Lidocaine hydrochloride, and Hydroxyethylcellulose,

In one embodiment of the present invention, the relative weights of these ingredients are 78%, 10%, 5%, 4% and 2%, respectively.

In another embodiment of the present invention, the formulation also includes lemon oil, chamomile extract, comfrey extract, mulberry extract and licorice extract, each in amount of approximately less than one-of one percent, by weight.

In another embodiment of the present invention, the method also includes the step of protecting the tattooed area with a sterile bandage after application of the formulation.

In another embodiment of the present invention, the skin covering the tattoo is opened using a tattoo needle attached to a standard tattoo machine.

In another embodiment of the present invention, the tattoo needle used to open the skin is a size 14 round needle.

In another embodiment of the present invention, as the skin covering the tattoo is opened, it is treated an anesthetic introduced through the tattoo needle.

In another embodiment of the present invention, the formulation is allowed to remain on the tattooed area for 10 minutes prior to removal.

Various other features and advantages of the present invention should become readily apparent with reference to the following detailed description.

### DETAILED DESCRIPTION

The present invention provides a formulation and method for removing tattoos. As used herein, “tattooed area” refers to any area of skin that includes tattoo ink. The method of the present invention may be performed to remove a mature tattoo. A mature tattoo is defined herein as a tattoo in which most of the tattoo ink pigment particles have been engulfed by, and reside in the cytoplasm of, dermal cells such as, for example, macrophages and fibroblasts. Alternatively, the method of the present invention may be performed to remove freshly applied or immature tattoos. A freshly applied or immature tattoo may be less than one week old, for example, 24-72 hours old. In a freshly applied tattoo or an immature tattoo, a majority of the tattoo ink pigment particles remain free in the interstitial space between dermal cells. Histological analysis of freshly applied tattoos shows that the tattoo ink pigment particles remain in the free extracellular space of the dermal cells for several days before the pigment particles are engulfed by macrophages and/or fibroblast cells.

The first step in the process of the present invention involves sterilizing the tattooed area using a standard disinfectant such as an antibacterial soap or isopropyl alcohol. Any hair in that tattooed area should be removed prior to the sterilization process to prevent the introduction of bacteria. The tattooed area may be treated with a topical anesthetic such as lidocaine to minimize discomfort during the process.

Next, using a standard tattoo machine, the skin in the tattooed area is opened to access the layer of the ink using light pressure and a circular motion. In the preferred embodiment, a large shader tattoo needle such as a 14 Round is used to minimize discomfort and prevent scaring. A topical anesthetic may be introduced into the tattoo machine to allow application during the skin-opening process. The entirety of the skin covering the tattooed area is treated in this manner to allow access to all ink making up the tattoo. Once the tattooed area has been opened in this manner, the tattooed area is cleaned with water or an anesthetic liquid.

The next step is to apply a thin layer of the formulation of the present invention to the tattooed area in an amount to cover the opened skin. In the preferred embodiment of the present invention, the formulation is comprised of the following ingredients:

| Ingredient | Percentage (by weight) |
|---|---|
| Sea Salt | 78.00 |
| Purified Water (Aqua) | 10.00 |
| Aloe Barbadensis (Aloe Vera) Extract | 5.00 |
| Lidocaine Hydrochloride | 4.00 |
| Hydroxyethylcellulose | 2.00 |

It should be realized that the listed percentages of these ingredients are approximations. As will be understood by one of ordinary skill in the art, the formulation will not lose its efficacy as a result of slight variations made to the relative weights of the ingredients. The formulation may also contain the following ingredients, each in an amount of less than one percent, by weight: lemon oil, chamomile extract, comfrey extract, mulberry extract and licorice extract. These ingredients are used as moisturizers and skin-care additives and do not affect the efficacy of the process described herein.

Once applied, the formulation should be allowed to sit on the tattooed area for at least 10 minutes to allow for treatment of the tattoo ink. Then, the remainder of formulation is removed from the tattooed area using clean water and a sterile applicator such as gauze. The tattooed area will appear swollen as a result of the formulation being absorbed by the skin. The tattooed region should then be dried and covered with a non-stick dressing. Approximately 24-48 hours following treatment, a scab will form over the tattooed area, allowing exfoliation of the tattoo ink. The scab will protect the area and the product that has soaked into the opened skin. The product will draw out the unwanted ink into the scab over the following one to two weeks. Depending on a variety of factors such as the location of the tattooed area, the patient's natural skin pigment and the ink used in the tattoo to be removed, repeated treatments may be necessary.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A method for removing a tattoo comprising:
(i) opening at least a portion of a tattooed area; and
(ii) applying a formulation to the tattooed area, the formulation consisting essentially of: sea salt, purified water, aloe barbadensis (aloe vera) extract, lidocaine hydrochloride and hydroxyethylcellulose.

2. The method of claim 1 in which the formulation consists essentially of 78% sea salt, 10% purified water, 5% aloe barbadensis (aloe vera) extract, 4% lidocaine hydrochloride and 2% hydroxyethylcellulose.

3. The method of claim 1 wherein the formulation further contains lemon oil, chamomile extract, comfrey extract, mulberry extract and licorice extract, each in amount of approximately less than one-of one percent, by weight.

4. The method of claim 1 further comprising protecting the tattooed area with a sterile bandage after application of the formulation.

5. The method of claim 1 wherein the tattooed area is opened using a tattoo needle attached to a standard tattoo machine.

6. The method of claim 5 in which the tattoo needle is a size 14 round tattoo needle.

7. The method of claim 5 further comprising treating the tattooed area with an anesthetic introduced through the needle.

8. The method of claim 1 further comprising allowing the formulation to remain on the tattooed area for 10 minutes.
